(19) United States
(12) Patent Application Publication
(10) Pub. No.: US 2015/0078961 A1
(43) Pub. Date: Mar. 19, 2015

(54) STERILE BARRIER PACKAGING SYSTEM

(71) Applicant: David B. Opie, Severna Park, MD (US)

(72) Inventor: David B. Opie, Severna Park, MD (US)

(73) Assignee: NOXILIZER, INC., Baltimore, MD (US)

(21) Appl. No.: 14/239,583

(22) PCT Filed: Aug. 17, 2012
(86) PCT No.: PCT/US2012/051403

§ 371 Date: Apr. 23, 2014

Related U.S. Application Data
(60) Provisional application No. 61/525,427, filed on Aug. 19, 2011.

(51) Int. Cl. A61L 2/20; A61L 2/26

(52) U.S. Cl. CPC A61L 2/20; A61L 2/26; A61B 2019/0202
USPC 422/28; 206/366; 206/370

(57) ABSTRACT

A sterile barrier packaging system includes a container configured to hold at least one object to be sterilized, and a respective mating component for the object. The container is configurable in an expanded configuration, wherein the object and the mating component are held spaced apart in mutual registration. The container is further configurable in a compressed configuration, wherein the object and mating component are mated. A method of sterilizing an object includes sterilizing the object in a container holding the object and a mating component spaced apart in mutual registration and subsequently compressing the container to mate the component with the object.

### BACKGROUND OF THE INVENTION

This application claims priority to U.S. Provisional Patent Application 61/525,427 filed Aug. 19, 2011, which is incorporated by reference in its entirety herein.

1. Field

This application relates generally to sterilization packaging and more particularly to sterilization packaging configured to allow sterilization prior to sealing of a product having a removable cap.

2. Description of Related Art

Sharp instruments to be packaged for sterilization may present particular technical challenges. Generally, such instruments are shipped with protective coverings or caps over the sharp portion in order to reduce damage to the products and/or package during shipping. The cap, however, tends to impede exposure of sterliant compositions during exposure. Similarly, in sterilization of pre-filled syringes, needles should be sterilized prior to capping with protective coverings. Cap materials may be selected such that they are permeable to sterilant gas, however this may tend to place limitations on both cap material and sterilant. For example, rubber caps are sufficiently permeable to ethylene oxide to allow for sterilization with the cap in place.

### BRIEF SUMMARY OF THE INVENTION

A sterile barrier packaging system includes a container configured to hold at least one object to be sterilized, and a respective mating component for the object. The container is configurable in an expanded configuration, wherein the object and the mating component are held spaced apart in mutual registration. The container is further configurable in a compressed configuration, wherein the object and mating component are mated. A method of sterilizing an object includes sterilizing the object in a container holding the object and a mating component spaced apart in mutual registration and subsequently compressing the container to mate the component with the object.

### BRIEF DESCRIPTION OF THE DRAWINGS

FIG. 1 is a figure illustrating a typical package containing several syringes;

FIG. 2 is a cross sectional side view of a tub in accordance with an embodiment of the present invention in an expanded configuration; and

FIG. 3 is a cross sectional side view of a tub in accordance with an embodiment of the present invention in a compressed configuration.

### DETAILED DESCRIPTION OF THE INVENTION

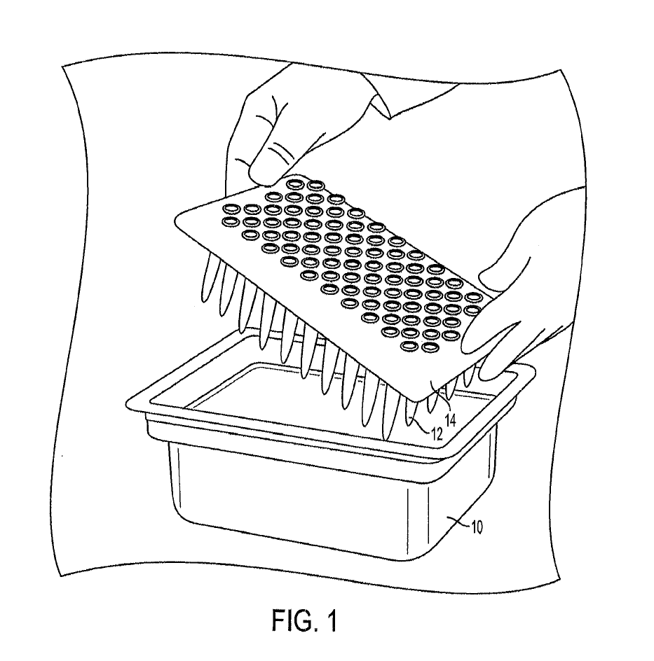

Referring now to FIG. 1, an example of a product or object to be sterilized is shown. A container, or tub, 10 is arranged to hold a tray supporting multiple prefillable syringes 12. In a typical example, the tub may hold 100 syringes that are ready to be filled. The syringes are supported by a plate 14 that includes holes configured to allow a barrel of each syringe to pass through while being supported at an upper end. The plate may be, for example, polypropylene, and the tub may be polystyrene. The tub may include a barrier layer (such as, e.g., Tyvek®) that permits gases to enter and exit the package through this barrier layer and serves as a barrier to microbes and dirt, protecting the tub contents. Similarly, the tub may be sealed with a barrier lid, not shown. Each tub may be placed in a pouch to further protect the tub and syringes.

In a sterilization method in accordance with an embodiment, the tub 10 and the syringes 12 contained therein are sterilized after the syringes are loaded into the tub and the tub is sealed. The sterilization method may make use of NO₂, ethylene oxide, hydrogen peroxide, or another sterilant gas.

As described above, the complexity or orientation of product components may hinder easy sterilization of the product while the seal is in place. In this case, it may be useful to allow for manipulation of the products after sterilization, without removing them from their sterile barrier packaging system, in order to place them in the proper orientation for shipping and use.

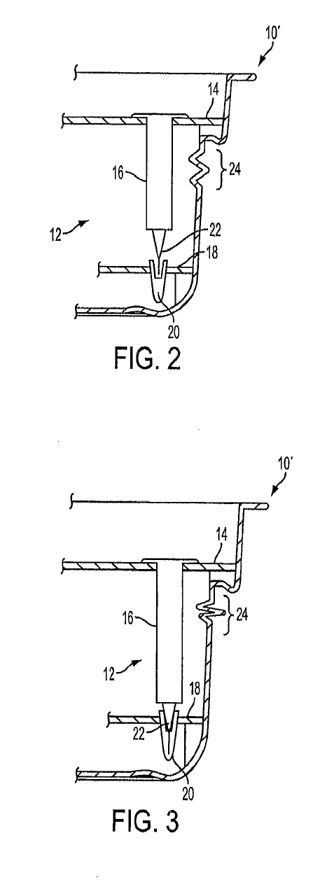

An embodiment of a package 10′ that addresses this concern is illustrated in FIGS. 2 and 3. The package 10′ includes the plate 14 that supports the barrels 16 of the syringes 12. A lower plate 18 supports the respective caps 20 for each syringe 12. A needle portion 22 of the syringe 12 is shown suspended above the cap 20 in FIG. 2. A portion 24 of the side of the package 10′ is formed in an accordion shape, allowing the package 10′ to be compressible.

As will be appreciated, compressibility does not depend on the existence of an accordion shape, and alternate compressible structures may be employed. For example, slidably mated telescoping wall portions may be used, though such an arrangement may tend to be more difficult to seal. Similarly, a flexible material may be used, allowing relative motion between a top and a bottom without use of either telescoping or accordion folding. In any of these embodiments, guide pins or other aligning structure may be used, extending generally between the plate 14 and the lower plate 18 to reduce lateral motion of the package during compression. External alignment structure may similarly be employed.

The package 10′ is shown in FIG. 2 in an expanded configuration. In this configuration, the needle 22 is suspended above the cap 20, allowing sterilant to flow around the needle and inside the cap. As shown in the Figure, the cap may be separated from the syringe by a relatively small distance, as long as gas flow is possible. Where the distance is relatively small, it is possible to pulse sterilant gas and to use vacuum purging to ensure that sterilant contacts all relevant surfaces. The two plates ensure proper registration between the syringes and respective caps.

FIG. 3 illustrates a collapsed configuration, wherein the accordion portion 24 is compressed. As a result, the needle 22 becomes firmly engaged with the cap 20.

In an embodiment, the package remains collapsed once the caps are attached. In an alternate approach, the package resiliently returns to its expanded configuration. In this second approach, it will be appreciated that the caps should be sufficiently engaged with the syringes that they do not disengage on expansion.

In alternate approaches, the caps may release from the lower support plate 18 after being mated to the syringes, or they might stay engaged with the lower plate.

In an embodiment, caps that are intended to mate to a threaded luer hub may be designed so that the caps may be applied to the fluid path without twisting (pressed into place with a linear motion), but are then removed with a twisting motion.

While many of the concepts are described for syringes, these concepts can be applied to many applications including, but not limited to surgical tools such as drill bits, saw and/or knife blades, medical implants, and the like.

Although the invention has been described in detail for the purpose of illustration based on what are currently considered to be the most practical and preferred embodiments, it is to be understood that such detail is solely for that purpose and that the inventions are not limited to the disclosed embodiments, but, on the contrary, are intended to cover modifications and equivalent arrangements that are within the spirit and scope of the described embodiments. For example, it is to be understood that the present invention contemplates that, to the extent possible, one or more features of any embodiment can be combined with one or more features of any other embodiment.

1. A method of sterilizing an object comprising:
sterilizing the object in a container holding the object and a respective mating component while holding the object and the mating component spaced apart in mutual registration; and
compressing the container to bring the object and its mating component into a mated configuration.

2. A method as in claim 1, wherein the sterilizing comprises exposing the object to a sterilant gas.

3. A method as in claim 2, wherein the container is permeable to the sterilant gas.

4. A method as in any of the preceding claims claim 1, wherein the container is a tub configured to hold a plurality of syringes and respective caps for each syringe such that each syringe comprises an object to be sterilized and each cap comprises a respective mating component for each syringe.

5. A method as in claim 1, wherein the sterilant gas is NO₂.

6. A container for sterilizing an object comprising:
an object support, configured and arranged to support the object to be sterilized within the container;
a mating component support, configured and arranged to support a component that is adapted to mate with at least a portion of the object to be sterilized;
the container being configurable in an expanded configuration, wherein the object and the mating component are held spaced apart in mutual registration, and in a compressed configuration, wherein the object and the mating component are mated.

7. A container as in claim 6, wherein the object support comprises a support plate having a plurality of support structures, each support structure sized and located to support a respective object of a plurality of objects to be sterilized.

8. A container as in claim 7, wherein the mating component support comprises a plurality of support structures, each support structure sized and located to support a respective mating component for each object to be sterilized.

9. A container as in claim 6, wherein the object comprises a syringe and the mating component comprises a cap.

10. A container as in claim 6, wherein a sidewall of the container comprises an accordion section configured and arranged to allow the container to be configurable in the expanded and compressed configuration.

11. A container as in claim 6, wherein a sidewall of the container comprises a telescoping section configured and arranged to allow the container to be configurable in the expanded and compressed configuration.

12. A container as in claim 6, wherein at least a portion of the container comprises a material permeable to a sterilant gas.

13. A container as in claim 12, wherein the sterilant gas is NO₂.

14. A container as in claim 6, wherein the object is selected from the group consisting of: a syringe, a saw blade, a drill bit, and a knife blade, and wherein the mating component comprises a protective cap configured and arranged to protect a sharp portion of the object.

15. A container as in claim 6, further comprising a guiding structure, configured and arranged to constrain lateral relative movement between the object and the mating component during a re-configuration from the expanded configuration to the compressed configuration.
